(19) United States
(12) Patent Application Publication
(10) Pub. No.: US 2016/0289139 A1
(43) Pub. Date: Oct. 6, 2016

(54) PROCESSES FOR PRODUCING LINEAR ALKYLBENZENES

(71) Applicant: UOP LLC, Des Plaines, IL (US)

(72) Inventors: Lance A. Baird, Prospect Heights, IL (US); Daniel Ellig, Arlington Heights, IL (US)

(21) Appl. No.: 14/674,702

(22) Filed: Mar. 31, 2015

(51) Int. Cl. C07C 2/66; C07C 303/26; C07C 4/14; C10G 1/02; C07C 1/20; C07C 5/333

(52) U.S. Cl. CPC C07C 2/66; C07C 1/20; C07C 5/333; C07C 4/14; C10G 1/02; C07C 303/26

(57) ABSTRACT

One or more process for the production of a linear alkylbenzenes which can be used to produce surfactants. The linear hydrocarbon and the aromatic hydrocarbon are both produced from a renewable feedstock. The linear hydrocarbon may be produced by deoxygenating a natural oil and dehydrogenating the resulting hydrocarbon to provide an olefinic hydrocarbon. The aromatic hydrocarbon may be produced from the pyrolysis of a biomass and the deoxygenation of a pyrolysis oil.

### FIELD OF THE INVENTION

This invention relates generally to processes for the manufacture of linear alkylbenzenes, and more particularly to processes for the manufacture of linear alkylbenzenes from renewable feedstocks.

### BACKGROUND OF THE INVENTION

Linear alkylbenzenes (LAB) are organic compounds with the formula C₆H₅CₙH₂ₙ₊₁. While “n” can have any practical value, current commercial use of alkylbenzenes requires that n lie in the range of 10 to 16, or in the range of 8 to 15, or in the range of 10 to 13, or in the range of 12 to 15, or in the range of 9 to 14. These specific ranges are often required when the alkylbenzenes are used as intermediates in the production of surfactants for detergents. Because the surfactants created from alkylbenzenes are biodegradable, the production of alkylbenzenes has grown rapidly since their initial uses in detergent production in the 1960s. The linearity of the paraffin chain in the alkylbenzenes is key to the material's biodegradability and effectiveness as a detergent. A major factor in the final linearity of the alkylbenzenes is the linearity of the paraffin component.

While detergents made utilizing alkylbenzene-based surfactants are biodegradable, many processes for creating alkylbenzenes are not entirely based on renewable sources. Specifically, alkylbenzenes are currently produced from kerosene originating from petroleum sources extracted from the earth. U.S. Pat. Nos. 3,950,448 and 5,276,231 both disclose processes from the production of linear alkylbenzenes from fossil fuel feedstocks. In these processes, linear hydrocarbons and benzene may be combined to form the linear alkylbenzenes. The linear hydrocarbons and benzene may be derived from the processing of petroleum crude oil. However, due to the cost and limited supply of the petroleum crude oil, there is an interest in producing chemicals, such as linear alkylbenzenes, from feedstocks other than from petroleum crude oil due to the cost and limited supply of the petroleum crude oil.

Accordingly, various advances have resulted in processes which produce linear hydrocarbons from renewable feedstocks having glycerides and fatty acids, and the linear hydrocarbon may be utilized to produce linear alkylbenzenes. While the linear portion of the linear alkylbenzenes may be produced from a renewable feedstock, the linear alkylbenzenes still require an aromatic hydrocarbon source that is derived from a petroleum crude oil source. Accordingly, the linear alkylbenzenes are not completely independent of petroleum based crude oil source

Therefore, it would be desirable to have one or more processes for producing linear alkylbenzenes that utilize renewable feedstocks for the production of the aromatic portion and renewable feedstocks for the production of the linear hydrocarbon portion.

### SUMMARY OF THE INVENTION

One or more processes have been invented in which both the linear component of the linear alkylbenzenes and the aromatic component of the linear alkylbenzenes are produced from a renewable feedstock.

Therefore, in a first embodiment of the invention, the present invention may be characterized broadly as providing a process for generating an alkylbenzene product by: producing a linear hydrocarbon from a renewable triglyceride feedstock; producing an aromatic hydrocarbon from a biomass feedstock; and, reacting the linear hydrocarbon produced from the renewable triglyceride feedstock and the aromatic hydrocarbon produced from the biomass feedstock to produce an alkylbenzene product.

In one or more embodiments of the present invention, the linear hydrocarbon is produced by deoxygenating the renewable triglyceride feedstock to provide a deoxygenated linear hydrocarbon. It is contemplated that the deoxygenated linear hydrocarbon is dehydrogenated to provide a linear olefin hydrocarbon, and wherein the linear olefin hydrocarbon is reacted with the aromatic hydrocarbon to produce the alkylbenzene product.

In some embodiments of the present invention, the linear hydrocarbon comprises a linear olefin hydrocarbon.

In various embodiments of the present invention, the aromatic hydrocarbon is produced from the biomass feedstock by pyrolysis.

In many embodiments of the present invention, the pyrolysis produces a pyrolysis oil. It is contemplated that the pyrolysis oil is deoxygenated under conditions to maintain aromatic hydrocarbons and to provide a benzene rich aromatic stream. It is further contemplated that the benzene rich aromatic stream is reacted with the linear hydrocarbon produced from the renewable feedstock.

In a second embodiment of the invention, the present invention may be characterized broadly as providing a process for generating an alkylbenzene product from renewable resources by: deoxygenating a renewable triglyceride feedstock in a deoxygenation zone having a catalyst and being operated under deoxygenation conditions to provide a paraffin hydrocarbon stream; dehydrogenating the paraffin hydrocarbon stream in a dehydrogenation zone to provide an olefin hydrocarbon stream; producing an aromatic hydrocarbon stream from a biomass feedstock; and, alkylating the aromatic hydrocarbon stream with the olefin hydrocarbon stream in an alkylation zone having an alkylation catalyst and being operated under alkylation conditions to provide an alkylbenzene product stream.

In one or more embodiments of the present invention, the process further includes producing the aromatic hydrocarbon stream from the biomass feedstock by pyrolysis of the biomass feedstock to provide a pyrolysis oil. It is contemplated that the process also includes deoxygenating the pyrolysis oil and then separating an aromatic hydrocarbon stream from the deoxygenated pyrolysis oil.

In some embodiments of the present invention, the process further includes converting the pyrolysis oil into an aromatic rich hydrocarbon stream by contacting the pyrolysis oil with hydrogen in a reaction zone having a catalyst and being operated under hydroprocessing conditions to provide an aromatic rich hydrocarbon stream. It is contemplated that the process includes increasing the concentration of benzene in the aromatic rich hydrocarbon stream by dealkylating the aromatic rich hydrocarbon stream.

In one or more embodiments of the present invention, the process includes sulfonating the alkylbenzene product stream to provide a surfactant product.

In yet a third embodiment of the invention, the present invention may be characterized broadly as providing a process for generating an alkylbenzene product from renewable resources by: producing a linear hydrocarbon stream from a triglyceride renewable feedstock; pyrolyzing a biomass feedstock to provide a pyrolysis oil; hydrogenating the pyrolysis oil to provide a deoxygenated effluent; separating an aromatic hydrocarbon stream from the deoxygenated effluent, the aromatic hydrocarbon stream comprising benzene; and, alkylating the aromatic hydrocarbon stream with the linear hydrocarbon stream from the renewable feedstock in an alkylation zone having an alkylation catalyst and being operated under alkylation conditions to provide an alkylbenzene product stream.

In one or more embodiments of the present invention, the process includes pretreating the pyrolysis oil to remove contaminants from the pyrolysis oil prior to deoxygenating the pyrolysis oil with hydrogen in a reaction zone having a catalyst and being operated under hydroprocessing conditions to provide an aromatic rich hydrocarbon stream. It is contemplated that the pretreatment comprises at least one of a filtration and an ion exchange treatment.

In some embodiments of the present invention, the process includes producing a linear hydrocarbon stream from a triglyceride renewable feedstock by deoxygenating a renewable feedstock in a deoxygenation zone having a catalyst and being operated under deoxygenation conditions to provide a paraffin hydrocarbon stream. It is contemplated that the process includes dehydrogenating the paraffin hydrocarbon stream in a dehydrogenation zone to provide an olefin hydrocarbon stream, the olefin hydrocarbon stream alkylating the aromatic hydrocarbon stream in the alkylation zone. It is also contemplated that the process includes sulfonating the alkylbenzene product stream to provide a surfactant product.

Additional embodiments, aspects, and details of the invention, which may be combined in any manner, are set forth in the following detailed description of the invention.

### DETAILED DESCRIPTION OF THE DRAWINGS

One or more exemplary embodiments of the present invention will be described below in conjunction with the following drawing figures, in which:

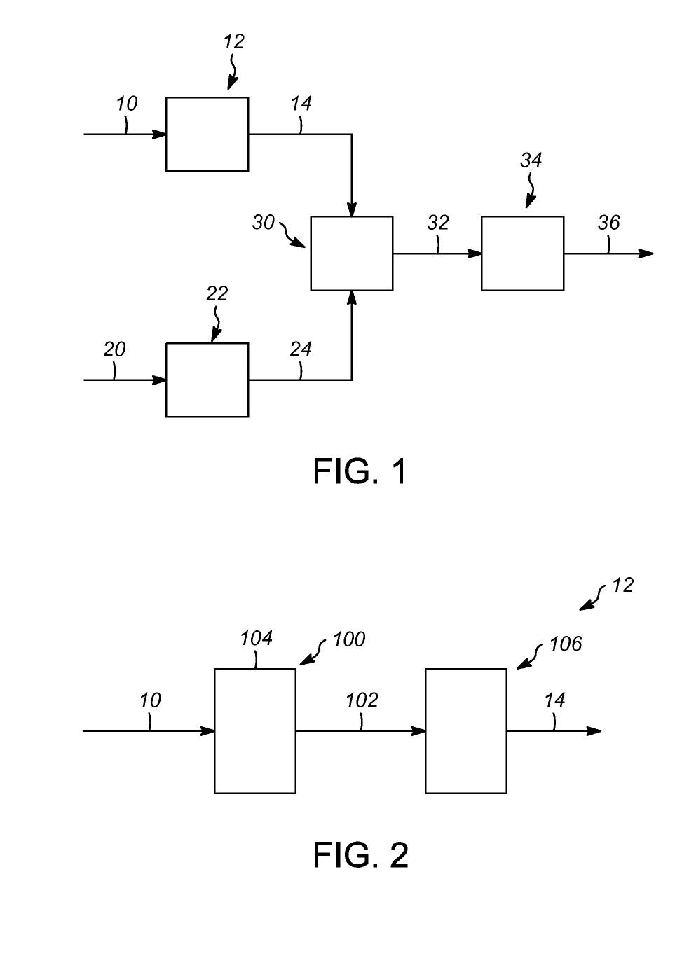

FIG. 1 shows a process flow diagram according to one or more embodiments of the present invention;

FIG. 2 shows a process flow diagram for producing a linear hydrocarbon for use in one or more embodiments of the present invention; and,

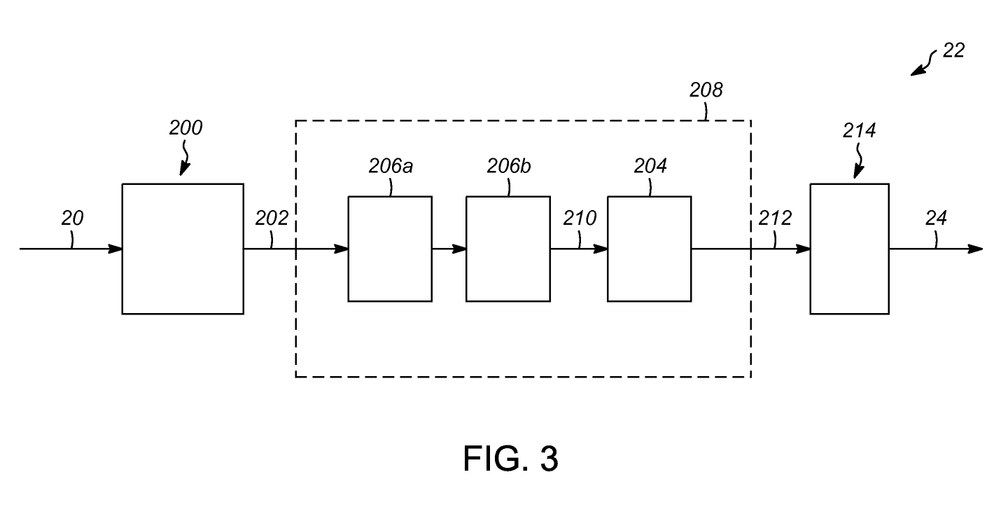

FIG. 3 shows a process flow diagram for producing an aromatic hydrocarbon for use in one or more embodiments of the present invention.

### DETAILED DESCRIPTION OF THE INVENTION

As mentioned above, one or more processes have been invented in which both the linear components and the aromatic components of linear alkylbenzenes are produced from a renewable resource. By utilizing renewable sources for both components, the production of linear alkylbenzenes would not be dependent on fossil fuels such as petroleum crude oil or the like. Thus, the linear alkylbenzenes could be produced from renewable resources and be used to make surfactants for using in detergents and cleaning formulations.

As used in the present disclosure, the terms “renewably-based” or “renewable” denote that the carbon content of the renewable hydrocarbon (paraffins, olefins, aromatics, alkylbenzene, linear alkylbenzene or subsequent products prepared from renewable hydrocarbons), is from a “new carbon” source as measured by ASTM test method D6866-05, “Determining the Bio-based Content of Natural Range Materials Using Radiocarbon and Isotope Ratio Mass Spectrometry Analysis”, hereby incorporated by reference in its entirety. This test method measures the ¹⁴C/¹²C isotope ratio in a sample and compares it to the ¹⁴C/¹²C isotope ratio in a standard 100 mass % bio-based material to give percent bio-based content of the sample. Additionally, “bio-based materials” are organic materials in which the carbon comes from recently, on a human time scale, fixated carbon dioxide present in the atmosphere using sunlight energy, photosynthesis. On land, this carbon dioxide is captured or fixated by plant life such as agricultural crops or forestry materials. In the oceans, the carbon dioxide is captured or fixated by photosynthesizing bacteria or phytoplankton. For example, a bio-based material has a ¹⁴C/¹²C isotope ratio greater than 0. Contrarily, a fossil-based material has a ¹⁴C/¹²C isotope ratio of about 0. The term “renewable” with regard to compounds such as hydrocarbons (paraffins, olefins, di-olefins, aromatics, alkylbenzene, linear alkylbenzene etc.) also refers to compounds prepared from biomass using thermochemical methods such as (e.g., Fischer-Tropsch catalysts), biocatalysts (e.g., fermentation), or other processes, for example.

A small amount of the carbon atoms in the atmospheric carbon dioxide is the radioactive isotope ¹⁴C. This ¹⁴C carbon dioxide is created when atmospheric nitrogen is struck by a cosmic ray generated neutron, causing the nitrogen to lose a proton and form carbon of atomic mass 14 (¹⁴C), which is then immediately oxidized, to carbon dioxide. A small but measurable fraction of atmospheric carbon is present in the form of ¹⁴C. Atmospheric carbon dioxide is processed by green plants to make organic molecules during the process known as photosynthesis. Virtually all forms of life on Earth depend on this green plant production of organic molecules to produce the chemical energy that facilitates growth and reproduction. Therefore, the ¹⁴C that forms in the atmosphere eventually becomes part of all life forms and their biological products, enriching biomass and organisms which feed on biomass with ¹⁴C. In contrast, carbon from fossil fuels does not have the signature ¹⁴C/¹²C ratio of renewable organic molecules derived from atmospheric carbon dioxide. Furthermore, renewable organic molecules that biodegrade to carbon dioxide do not contribute to an increase in atmospheric greenhouse gases as there is no net increase of carbon emitted to the atmosphere.

Assessment of the renewably based carbon content of a material can be performed through standard test methods such as using radiocarbon and isotope ratio mass spectrometry analysis. ASTM International (formally known as the American Society for Testing and Materials) has established a standard method for assessing the bio-based content of materials. The ASTM method is designated ASTM-D6866.

The application of ASTM-D6866 to derive “biobased content” is built on the same concepts as radiocarbon dating, but without use of the age equations. The analysis is performed by deriving a ratio of the amount of radiocarbon (¹⁴C) in an unknown sample compared to that of a modern reference standard. This ratio is reported as a percentage with the units “pMC” (percent modern carbon). If the material being analyzed is a mixture of present day radiocarbon and fossil carbon, which contains very low levels of radiocarbon, then the pMC value obtained correlates directly to the amount of biomass material present in the sample.

A renewable linear alkylbenzene product is provided which has the general chemical formula C₆H₅CₙH₂ₙ₊₁ of which the carbon is predominantly modern carbon, as defined and measured by ASTM D6866, and not derived from petroleum, and has a linearity of the paraffin alkyl group preferably of greater than 80 mass % but more preferably greater than 90 mass % and most preferably of at least 92 mass %. Thus the alkylbenzene product contains at least 80 mass % of linear alkylbenzenes meaning alkylbenzenes wherein the paraffin alkyl group is a linear paraffin alkyl group. The renewable linear alkylbenzene consists of a benzene ring which comprises the portion of the chemical formula of C₆H₅ that is alkylated with linear paraffin which is described by the CₙH₂ₙ₊₁ portion of the formula. Often, linear alkylbenzenes are sulfonated to produce a linear alkylbenzene sulfonate as a surfactant for use in detergents. For the purposes of use as material to produce a linear alkylbenzene sulfonate it is preferable that the paraffins carbon chain length (n in the chemical formula) of the alkyl group is in the range of 10 to 16, or in the range of 8 to 15, or in the range of 10 to 13, or in the range of 12 to 15, or in the range of 9 to 14. The carbons on the paraffin chain will be rich in ¹⁴C relative to that seen in petroleum derived paraffins. Carbon enriched in ¹⁴C is generally considered modern carbon, atmospheric carbon, or new carbon and is an indicator of how much renewable carbon is in the compound. Analytical methods such as ASTM D6866, as discussed above, can be used to determine the amount of carbon content that is modern carbon and not derived from petroleum by analyzing the amount of ¹⁴C in the compound.

The linearity of alkylbenzene product is mostly dependent on the linearity of the paraffins used to alkylate the benzene. It is a common rule of thumb by those skilled in the art that the linearity of a paraffin feed drops by about 5-7 mass % after dehydrogenation and alkylation. Therefore paraffin with 97 mass % linearity (or alternatively 3 mass % isoparaffin) would result in an alkylbenzene product with linearity around 90-92 mass %. This sets the requirement for paraffin linearity about 5-7 mass % higher than the specification for the alkylbenzene product. Typically the linearity of the paraffin product is measured by UOP 621, UOP 411, or UOP 732 standard test method available from ASTM, which is hereby incorporated by reference in its entirety. Linear alkylbenzenes may be analyzed using ASTM Standard Test Method D4337 hereby incorporated by reference in its entirety.

Contrary to current theory, hydrocracking to a lower carbon chain length from a longer chain length is a very inefficient way to produce normal paraffins. For example, most plant oils have predominantly C₁₆ and C₁₈ carbon chains. If C₁₀ to C₁₃ carbon chains are required to make the desired alkylbenzene product, one might believe that cracking the hydrodeoxygenated C₁₆ and C₁₈ normal paraffins and the decarboxylated/decarbonylated C₁₅ and C₁₇ normal paraffins to the C₁₀ to C₁₃ range would be a suitable route. However, hydrocracking results primarily in branched paraffins so very little of a hydrocracked product material would result in normal paraffins. Therefore to produce linear paraffins in the desired carbon number ranges for linear alkylbenzene it is highly preferable to use oil with large amounts of C₁₀, C₁₂ and C₁₄ carbon chain length fatty acids, these oils include coconut oil, palm kernel oil, and babassu oil.

With these general principles in mind, one or more embodiments of the present invention will be described with the understanding that the following description is not intended to be limiting.

As shown in FIG. 1, a first renewable feedstock 10 is passed to a linear conversion zone 12 to produce a linear hydrocarbon stream 14. An exemplary linear conversion zone 12 is described below in relation to FIG. 2. The first renewable feedstock 10 preferably comprises a triglyceride feedstock meaning that the feedstock comprises triglycerides, diglycerides, monoglycerides, FFAs, and mixtures thereof. In an exemplary embodiment, the linear hydrocarbon stream 14 comprises olefinic hydrocarbons, most preferably, depending on the catalyst, between 4 to 18 wt % mono-olefins, or between 7 to 12 wt % mono-olefins, or about, 10% mono-olefins. A second renewable feedstock 20 is passed to an aromatic conversion zone 22 to produce an aromatic hydrocarbon stream 24. An exemplary aromatic conversion zone 22 is described below in relation to FIG. 3. Preferably, the aromatic hydrocarbon stream 24 comprises a benzene rich stream.

The linear hydrocarbon stream 14 and the aromatic hydrocarbon stream 24 are both passed to a reaction zone 30 which is configured to produce a linear alkylbenzene product stream 32. The reaction zone 30 may comprise an alkylation zone, which is described in more detail in U.S. Pat. Nos. 3,950,448 and 5,276,231, the entirety of both of which are incorporated herein by reference. The linear alkylbenzene product stream 32 may be passed to a sulfonation zone 34 in which the linear alkylbenzene will be sulfonated and neutralized with sodium hydroxide to produce a surfactant product stream 36 in which both the aromatic hydrocarbon and the linear hydrocarbon of the linear alkylbenzenes used to produce the surfactant were produced from a renewable feedstock.

Turning to FIG. 2, an exemplary linear conversion zone 12 is depicted for converting the first renewable feedstock 10 into the linear hydrocarbon stream 14. The first renewable feedstock 10 preferably includes any of those which comprise glycerides and free fatty acids (FFA). Examples of these feedstocks include, but are not limited to, canola oil, corn oil, soy oils, rapeseed oil, soybean oil, colza oil, tall oil, sunflower oil, hempseed oil, olive oil, linseed oil, coconut oil, castor oil, peanut oil, palm oil, including palm fatty acid distillate, mustard oil, cottonseed oil, tallow, yellow and brown greases, lard, train oil, fats in milk, fish oil, algal oil, sewage sludge, cuphea oil, camelina oil, jatropha oil, curcas oil, babassu oil, palm kernel oil, crambe oil, and the like. Biorenewable is another term used to describe these feedstocks. As mentioned above, the glycerides, FFAs, and fatty acid alkyl esters, of the typical vegetable oil or animal fat contain aliphatic hydrocarbon chains in their structure which have about 8 to about 24 carbon atoms with a majority of the oils containing high concentrations of fatty acids with 16 and 18 carbon atoms.

In various embodiments of the present invention, the linear conversion zone 12 includes a deoxygenation zone 100 which contains a catalyst and which is configured to deoxygenate the glycerides and free fatty acids in the first renewable feedstock 10 and provide a linear paraffin stream 102. Such a deoxygenation zone 100 is disclosed for example in U.S. Pat. No. 8,039,682, the entirety of which is incorporated herein by reference. In general, the deoxygenation zone 100 includes one or more reactors 104 comprising a suitable catalyst(s) for promoting deoxygenation reactions and which may be any of those well known in the art such as nickel or nickel/molybdenum dispersed on a high surface area support. Other catalysts include one or more noble metal catalytic elements dispersed on a high surface area support. Non-limiting examples of noble metals include Pt and/or Pd dispersed on gamma-alumina. Generally, deoxygenation conditions include a temperature of about 40 to about 700° C. (104 to 1,292° F.) and a pressure of about 700 to about 21 MPa (100 to 3000 psig). Other operating conditions for the deoxygenation zone 100 are well known in the art.

The linear paraffin stream 102 may comprise mostly paraffinic hydrocarbons and accordingly, may be passed to a dehydrogenation zone 106 to convert the paraffinic hydrocarbons, in the presence of a suitable catalyst, into olefinic hydrocarbons. Such a process in disclosed in for example, U.S. Pat. No. 5,300,715, the entirety of which is incorporated herein by reference. The catalysts used for dehydrogenation are not critical to the processes of this invention. Many types of dehydrogenation catalysts are known as exemplified by U.S. Pat. Nos. 3,274,287; 3,315,007; 3,315,008; 3,745,112; and 4,430,517. Often the dehydrogenation catalysts are platinum group metal containing catalysts. One such catalyst is a layered composition comprising an inner core and an outer layer bonded to the inner core, where the outer layer comprises a refractory inorganic oxide having uniformly dispersed thereon at least one platinum group (Groups 8-10 of the periodic table) metal and at least one promoter metal, and where at least one modifier metal is dispersed on the catalyst composition. The dehydrogenation may be conducted in the liquid phase or in a mixed vapor-liquid phase, but preferably in the vapor phase. Typical dehydrogenation conditions involve a temperature of from about 400° to about 900° C. (752 to 1652° F.) and preferably from about 420 to about 550° C. (788 to 1022° F.). Generally for normal paraffins, the lower the molecular mass the higher the temperature required for comparable conversion. Pressures are generally from about 1 to about 1000 kPa(g) (0.15 to 145 psi(g)), preferably between about 100 and 400 kPa(g) (14.5 to 58.0 psi(g)), and a LHSV of from about 0.1 to about 100 hr⁻¹. As used herein, the abbreviation “LHSV” means liquid hourly space velocity, which is defined as the volumetric flow rate of liquid per hour divided by the catalyst volume, where the liquid volume and the catalyst volume are in the same volumetric units. If desired, the dehydrogenation product may be subjected to selective hydrogenation to convert diolefins to mono-olefins. U.S. Pat. No. 5,276,231, for instance, discloses the selective hydrogenation of diolefinic by-products from dehydrogenation.

From the dehydrogenation zone 106, the linear hydrocarbon stream 14 is provided and may be utilized as discussed above with respect to FIG. 1 to produce the linear alkylbenzene, and ultimately the surfactant product.

With reference to FIG. 3, an exemplary aromatic conversion zone 22 is shown in which the second renewable feedstock 20 is converted into the aromatic hydrocarbon stream 24. The second renewable feedstock 20 may comprise solid carbonaceous biomass feedstock, i.e., “biomass”, such as wood waste, agricultural waste, etc. which is passed to a pyrolysis zone 200 and subjected to pyrolysis, preferably fast pyrolysis. Fast pyrolysis is a thermal process during which the biomass is rapidly heated to pyrolysis temperatures of about 450 to about 600° C. (842 to 1,112° F.) in a substantially air free pyrolysis reactor. Under these conditions, a pyrolysis vapor stream including organic vapors, water vapor, and pyrolysis gases is produced, along with char (which includes ash and combustible hydrocarbon solids). The pyrolysis vapor maybe condensed to produce a pyrolysis oil. The biomass-derived pyrolysis oil is a complex organic liquid that typically contains about 40-50% hydrocarbon, 20-40% oxygen and 20-30% by weight water. The biomass-derived pyrolysis oil is also rich in aromatic compounds.

Biomass-derived pyrolysis oil is available from, for example, Ensyn Technologies Inc., of Ontario, Canada. The composition of biomass-derived pyrolysis oil is somewhat dependent on feedstock and processing variables. The biomass-derived pyrolysis oil may be produced, for example, from fast pyrolysis of wood biomass in a pyrolysis reactor. However, virtually any form of biomass can be considered for pyrolysis to produce biomass-derived pyrolysis oil. In addition to wood, biomass-derived pyrolysis oil may be derived from biomass material such as bark, agricultural wastes/residues, nuts and seeds, algae, grasses, forestry residues, cellulose and lignin, or the like. The biomass-derived pyrolysis oil may also be obtained by different modes of pyrolysis, such as fast pyrolysis, vacuum pyrolysis, catalytic pyrolysis, and slow pyrolysis (also known as carbonization) or the like.

A pyrolysis oil stream 202 may be passed to an upgrading zone 208 having one or more contaminant removal zones 206a, 206b and a hydroprocessing zone 204. The contaminant removal 206a, 206b zone may comprise a filtration zone 206a configured to remove particulates or other materials from the pyrolysis oil. Additionally, the contaminant removal zone may comprise an ion exchange zone 206b to remove metals from the pyrolysis oil. From the contaminant removal zones 206a, 206b, a purified pyrolysis oil stream 210 is passed to the hydroprocessing zone 204.

In the hydroprocessing zone 204, the pyrolysis oil in the purified pyrolysis oil stream 210 can be upgraded by removing the water and oxygen (i.e., deoxygenation) by contacting the pyrolysis oil with hydrogen and catalyst at suitable hydroprocessing conditions in one or more reactors. Such conditions are disclosed in for example, U.S. application Ser. Nos. 14/551,797 and 14/101,842 filed Nov. 24, 2014 and Dec. 10, 2013, respectively, and both of which are incorporated herein by reference. It is preferred that the hydroprocessing zone 204 is configured to maximize aromatic production, for example, by using two or more reactions and using a catalyst with a noble metal for a second stage hydrodeoxygenation. Preferably, the second reactor (or second stage) includes a higher temperature and lower pressure to shift the equilibrium and produce more aromatic hydrocarbons.

In a preferred embodiment, a first reactor (not shown) in the hydroprocessing zone 204 may include conditions include a reaction temperature of from about 100 to about 400° C. (212 to 752° F.), or from about 150 to about 350° C. (302 to 662° F.) in different embodiments. The reaction pressure is from about 2,000 to about 20,000 kPa (290 to 2,900 psi), or from about 3,400 to about 17,000 kPa (493 to 2,466 psi) in various embodiments. In this description, indicated pressures are gauge as opposed to absolute unless otherwise indicated. The LHSV is determined on a basis of weight of the first reactor feedstock/weight of deoxygenation catalyst/hour. The LHSV may be from about 0.10 to about 2 hr¹, and the required hydrogen to hydrocarbon ratio is about 1,000 to about 15,000 standard cubic feet of hydrogen per barrel of pyrolysis oil (SCF/B).

A second reactor (not shown) in the hydroprocessing zone 204 may operate with conditions for the simultaneous deoxygenation and dehydrogenation with the preservation of the aromatic compounds and include a temperature of from about 300 to about 540° C. (572 to 1,004° F.), or from about 400 to about 520° C. (752 to 968° F.), or from about 475 to about 520° C. (887 to 968° F.) in various embodiments. The pressure may be from about 340 to about 5,500 kPa (49 to 798 psi), or from about 1,000 to about 3,100 kPa (145 to 450 psi) in various embodiments. The LHSV may be from about 0.1 to about 3 hr¹. It is contemplated that a separation zone is disposed between reactors in hydroprocessing zone 204.

As shown in FIG. 3, an aromatic rich stream 212 may be passed from the upgrading zone 208, and more specifically the hydroprocessing zone 204 to a benzene extraction zone 214 configured to separate benzene from the other components. The benzene extraction zone 214 can include one or more vessels having adsorbents, and/or may comprise fractionation or distillation columns, all of which are preferably configured to separate benzene from the non-aromatics and the other aromatic hydrocarbons in the aromatic rich stream 212. Additionally, the benzene extraction zone 214 may include isomerization zones and trans/dealkylation zones configured to convert various aromatics into benzene to increase the benzene production yield from the benzene extraction zone 214.

The benzene extraction zone 214 will provide the aromatic hydrocarbon stream 24 which may be utilized as discussed above with respect to FIG. 1 to produce a linear alkylbenzene product, and ultimately the surfactant product.

In the various embodiments of the present invention, both the linear hydrocarbon and the aromatic hydrocarbon of the linear alkylbenzene may be produced from a renewable resource. Thus, the linear alkylbenzene and the surfactant produced therefrom are not dependent on fossil fuels/petroleum based feedstocks and can be made from renewable resources.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understanding the embodiments of the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for generating an alkylbenzene product comprising:
producing a linear hydrocarbon from a renewable triglyceride feedstock;
producing an aromatic hydrocarbon from a biomass feedstock;
reacting the linear hydrocarbon produced from the renewable triglyceride feedstock and the aromatic hydrocarbon produced from the biomass feedstock to produce an alkylbenzene product.

2. The process of claim 1 wherein the linear hydrocarbon is produced by deoxygenating the renewable triglyceride feedstock to provide a deoxygenated linear hydrocarbon.

3. The process of claim 2 wherein the deoxygenated linear hydrocarbon is dehydrogenated to provide a linear olefin hydrocarbon, and wherein the linear olefin hydrocarbon is reacted with the aromatic hydrocarbon to produce the alkylbenzene product.

4. The process of claim 1 wherein the linear hydrocarbon comprises a linear olefin hydrocarbon.

5. The process of claim 1 wherein the aromatic hydrocarbon is produced from the biomass feedstock by pyrolysis.

6. The process of claim 5 wherein the pyrolysis produces a pyrolysis oil.

7. The process of claim 6 wherein the pyrolysis oil is deoxygenated under conditions to maintain aromatic hydrocarbons and to provide a benzene rich aromatic stream.

8. The process of claim 7 wherein the benzene rich aromatic stream is reacted with the linear hydrocarbon produced from the renewable feedstock.

9. A process for generating an alkylbenzene product from renewable resources, the process comprising:
deoxygenating a renewable triglyceride feedstock in a deoxygenation zone having a catalyst and being operated under deoxygenation conditions to provide a paraffin hydrocarbon stream;
dehydrogenating the paraffin hydrocarbon stream in a dehydrogenation zone to provide an olefin hydrocarbon stream;
producing an aromatic hydrocarbon stream from a biomass feedstock; and,
alkylating the aromatic hydrocarbon stream with the olefin hydrocarbon stream in an alkylation zone having an alkylation catalyst and being operated under alkylation conditions to provide an alkylbenzene product stream.

10. The process of claim 9 further comprising:
producing the aromatic hydrocarbon stream from the biomass feedstock by pyrolysis of the biomass feedstock to provide a pyrolysis oil.

11. The process of claim 10 further comprising:
deoxygenating the pyrolysis oil and then separating an aromatic hydrocarbon stream from the deoxygenated pyrolysis oil.

12. The process of claim 11 further comprising:
converting the pyrolysis oil into an aromatic rich hydrocarbon stream by contacting the pyrolysis oil with hydrogen in a reaction zone having a catalyst and being operated under hydroprocessing conditions to provide an aromatic rich hydrocarbon stream.

13. The process of claim 12 further comprising:
increasing a concentration of benzene in the aromatic rich hydrocarbon stream by dealkylating the aromatic rich hydrocarbon stream.

14. The process of claim 9 further comprising:
sulfonating the alkylbenzene product stream to provide a surfactant product.

15. A process for generating an alkylbenzene product from renewable resources, the process comprising:
producing a linear hydrocarbon stream from a triglyceride renewable feedstock;
pyrolyzing a biomass feedstock to provide a pyrolysis oil;
hydrogenating the pyrolysis oil to provide a deoxygenated effluent;
separating an aromatic hydrocarbon stream from the deoxygenated effluent, the aromatic hydrocarbon stream comprising benzene; and,
alkylating the aromatic hydrocarbon stream with the linear hydrocarbon stream from the renewable feedstock in an alkylation zone having an alkylation catalyst and being operated under alkylation conditions to provide an alkylbenzene product stream.

16. The process of claim 15 further comprising:
pretreating the pyrolysis oil to remove contaminants from the pyrolysis oil prior to deoxygenating the pyrolysis oil with hydrogen in a reaction zone having a catalyst and being operated under hydroprocessing conditions to provide an aromatic rich hydrocarbon stream.

17. The process of claim 16 wherein pretreating the pyrolysis oil comprises at least one of a filtration and an ion exchange treatment.

18. The process of claim 16 further comprising:
producing a linear hydrocarbon stream from a triglyceride renewable feedstock by deoxygenating a renewable feedstock in a deoxygenation zone having a catalyst and being operated under deoxygenation conditions to provide a paraffin hydrocarbon stream.

19. The process of claim 18 further comprising:
dehydrogenating the paraffin hydrocarbon stream in a dehydrogenation zone to provide an olefin hydrocarbon stream, the olefin hydrocarbon stream alkylating the aromatic hydrocarbon stream in the alkylation zone.

20. The process of claim 19 further comprising:
sulfonating the alkylbenzene product stream to provide a surfactant product.
